(19) United States
(12) Patent Application Publication
(10) Pub. No.: US 2026/0071963 A1
(43) Pub. Date: Mar. 12, 2026

(54) Method for Obtaining Geological Information of a Soil Sample and Device Therefore

(71) Applicant: Royal Eijkelkamp B.V., Giesbeek (NL)

(72) Inventors: Marcus Hermanus Lichtenberg, Zutphen (NL); Hugo Jaap Eijkelkamp, Giesbeek (NL); Bob Klein Lankhorst, Amsterdam (NL)

(21) Appl. No.: 19/101,732

(22) PCT Filed: Jun. 14, 2023
(86) PCT No.: PCT/EP2023/065956

§ 371 Date: Feb. 6, 2025

(30) Foreign Application Priority Data
Aug. 10, 2022 (EP) 22189756.4

(51) Int. Cl. G01N 21/65; G01N 1/08; G01N 21/71; G01N 22/04; G01N 33/24

(52) U.S. Cl. CPC G01N 21/65; G01N 1/08; G01N 21/718; G01N 22/04; G01N 33/246

(57) ABSTRACT

A method for obtaining geological information of a soil sample including
providing a soil sampling tube having a tip portion, a top portion positioned opposite the tip portion and along the length, between the tip portion and the top portion, a cutaway portion extending along a substantial part of the length of the tube;
inserting the soil sampling tube into the ground to a predefined depth, while registering the insertion force in combination with the insertion depth of the tip portion;
while retracting the soil sampling tube with a soil sample out of the ground, analyzing the elements in the soil sample at discrete positions starting adjacent to the top portion end and moving towards the tip portion end of the soil sample tube;
and combining the analyzing results per discrete position starting adjacent to the top portion end and moving towards the tip portion end with the registered insertion force for the insertion depth of the tip portion starting from-the insertion to the predefined depth.

### CROSS-REFERENCE TO RELATED APPLICATIONS

This application is the United States national phase of International Patent Application No. PCT/EP2023/065956 filed Jun. 14, 2023, and claims priority to European Patent Application No. 22189756.4 filed Aug. 10, 2022, the disclosures of which are hereby incorporated by reference in their entireties.

### BACKGROUND OF THE INVENTION

### Field of the Invention

The invention relates to a method for obtaining geological information of a soil sample.

### Description of Related Art

Such a method is known from U.S. Pat. No. 7,692,789, which describes the insertion of a soil sampling tube having a C-shaped cross-section, such that a cutaway portion is provided. After insertion of the soil sampling tube to a predefined depth, the soil sampling tube is retracted from the ground taking along a soil sample enclosed by the soil sampling tube.

This soil sampling tube together with the soil sample is then transported off site to a laboratory, where the soil sample is analyzed at discrete positions by a laser induced breakdown spectroscope (LIBS) to determine the elements in the soil sample at the different discrete positions.

When the soil sampling tube together with the soil sample is to be transported off site to a laboratory, it is essential to preserve the soil sample as best as possible. For example, any moisture has the tendency to evenly distribute through the sample, especially when the pressure of surrounding soil is no longer present. The same applies for the consistency of the soil sample along the length of the sample. Loose layers would typically fall easily apart during transport and layers, which were pressurized by surrounding layers, can easily expand.

Furthermore, if at a specific site a number of soil samples are to be retrieved, then an equal number of soil sampling tubes is required. All of these sampling tubes need to be transported, which will increase costs substantially, apart from the specialized conditioning equipment required to condition each and every soil sample. However, this is not done in practice. Typically, the sample is just removed from the soil sampling tube and as a result the sample is completely disturbed.

### SUMMARY

It is an object of the invention to reduce or even remove the above-mentioned disadvantages.

This object can be achieved with the method according to an embodiment of the invention which method comprises the steps of:
providing a soil sampling tube having a tip portion, a top portion positioned opposite the tip portion and along the length, between the tip portion and the top portion, a cutaway portion extending along a substantial part of the length of the tube;
inserting the soil sampling tube into the ground to a predefined depth, while registering the insertion force in combination with the insertion depth of the tip portion;
while retracting the soil sampling tube with a soil sample out of the ground, analyzing the elements in the soil sample at discrete positions starting adjacent to the top portion end and moving towards the tip portion end of the soil sample tube;
combining the analyzing results per discrete position starting adjacent to the top portion end and moving towards the tip portion end with the registered insertion force for the insertion depth of the tip portion starting from the insertion to the predefined depth.

The insertion force is registered when the soil sampling tube is inserted into the ground. This already provides data on the consistency of the soil sample, which is to be retracted from the ground. Then by analyzing the elements in the soil sample at discrete positions as soon as the soil sample is retracted from the ground ensures that the soil sample is as undisturbed as possible and that for example moisture had no chance yet to spread through the sample.

Preferably, the soil sampling tube is rotated around the longitudinal axis at the predefined depth in order to break the sample loose from the ground and prevent loss of the sample, when the soil sampling tube is retracted.

Furthermore, the soil sample is analyzed as soon as the sample is retracted from the ground. This allows for the soil sampling tube to be emptied on site and a number of soil samples can be taken in a short period of time.

In a preferred embodiment of the method according to the invention the elements in the soil sample are analyzed using laser induced breakdown spectroscopy (LIBS) and/or Raman spectroscopy. These analyzing methods allow for a quick analysis at a discrete position of the soil sample, such that the soil sampling tube can be retracted relatively quick.

In yet a further preferred embodiment, an outer layer of the soil sample is removed before the soil sample is analyzed. This ensures that any disturbance of the soil sample by retracting the soil from the ground and/or rotating the soil sampling tube to break the sample loose from the ground has no influence on analyzing the elements in the soil sample.

In a further preferred embodiment, the elements are further analyzed by registering the gamma radiation for each discrete position.

The natural gamma radiation in the soil sample provides also an indication for the soil composition. This contributes is determining the geological information of the sample.

In yet a further embodiment, the soil sample is further analyzed by a microwave sensor for measuring the soil moisture.

A further embodiment of the invention further comprises the step of detecting the geographic position, combining the geographic position with the combined analyzing results and registered insertion force, and storing said combined information on a storage device.

Optionally, also the rotational orientation of the cutaway portion relative to the geographic position is registered. This rotational orientation could be used to determine in combination with other soil samples, the direction and thickness of a layer of an element in the ground.

By combining a plurality of soil samples with the corresponding geographic position, it is possible to generate a three-dimensional map of the geological structure of the ground, in which the plurality of soil samples was taken.

An embodiment of the invention also relates to a device for obtaining geological information of a soil sample according to the method of the invention, which device comprises:
a base with a support surface for positioning the device with the support surface onto a ground surface;
a soil sampling tube having a tip portion, a top portion positioned opposite the tip portion and along the length, between the tip portion and the top portion, a cutaway portion extending along a substantial part of the length of the tube;
guide means arranged to the base and along which the soil sampling tube is guided in a direction perpendicular to the support surface;
analyzing means arranged to the base, which analyzing means have a detecting area directed to the soil sampling tube.

The device can easily be positioned with the base on a spot on the ground. Then the soil sampling tube is inserted into the ground, while the insertion force is measured. At reaching the desired insertion depth the soil sampling tube is retracted from the ground and the soil sample is analyzed with the analyzing means.

In a preferred embodiment of the device according to the invention the cutaway portion of the soil sampling tube faces the detecting area of the analyzing means. Although the tube could be made of a material transparent for the analyzing means, a better accuracy is obtained, when the analyzing means can examine the soil sample without any obstructive layer in between.

In a further embodiment of the device according to the invention, means for removing an outer layer of the sample are arranged between the base and the analyzing means.

In a very preferred embodiment of the device according to the invention the analyzing means comprises at least one of a laser induced breakdown spectroscope (LIBS) and a Raman spectroscope. These spectroscopes allow for a quick determination of the elements in the soil sample for a specific discrete position.

Preferably, the analyzing means further comprise a gamma ray sensor. Natural gamma rays of the soil sample provide further geological information.

Furthermore, the analyzing means further comprise a microwave sensor for measuring the soil moisture.

Still a further embodiment of the device according to the invention further comprises a camera, preferably a UV sensitive camera, arranged to the base and directed to the cutaway portion of the soil sampling tube.

The images of the camera allow for a further visual inspection of the soil sample at a later time, without the need to preserve the soil sample. Furthermore, the images can be synced with the analyzing results.

### BRIEF DESCRIPTION OF THE DRAWINGS

The terms Fig., Figs., Figure, and Figures are used interchangeably in the specification to refer to the corresponding figures in the drawings.

These and other features of the inventions will be elucidated in conjunction with accompanying drawings.

FIG. 1 shows a schematic perspective view of an embodiment of a device according to the invention.

FIGS. 2A-2C show an embodiment of a method according to the invention.

### DESCRIPTION OF PREFERRED EMBODIMENTS

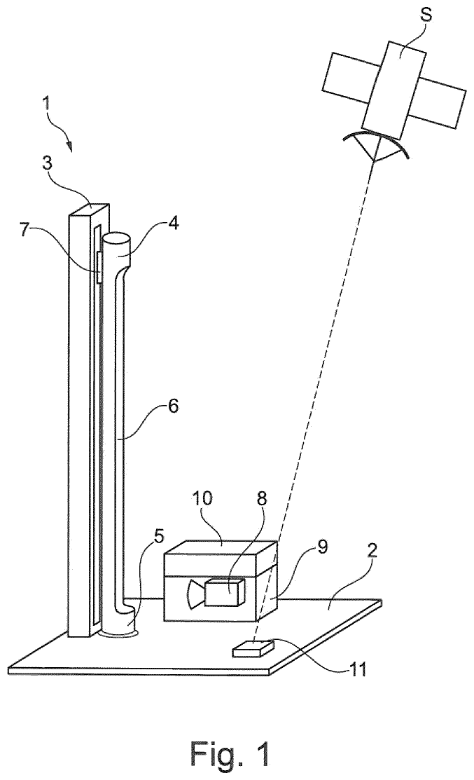

FIG. 1 shows an embodiment 1 of a device according to the invention. The device 1 has a base 2 formed by a plate. A guide column 3 is extending vertically from the base 2. A soil sampling tube 4, 5, 6 is arranged parallel and next to the guide column 3 and connected thereto via a connection 7.

The soil sampling tube 4, 5, 6 has a top portion 4, a tip portion 5 and a cutaway portion 6 extending along a substantial part of the length of the tube 4, 5, 6.

Next to the tube 4, 5, 6 a camera 8, a laser induced breakdown spectroscope (LIBS) 9 and a Raman spectroscope 10 are arranged on the base 2. These analyzing devices 8, 9, 10 are each directed to the cutaway portion 6 of the tube 4, 5, 6.

Furthermore, a GPS sensor 11 is arranged on the base 2 to receive a global positioning signal from a satellite S, such that the geographical position of the device 1 can be determined.

The device 1 is controlled by a controller, which controls the guide column 3, the camera 8, the laser induced breakdown spectroscope 9, and the Raman spectroscope 10.

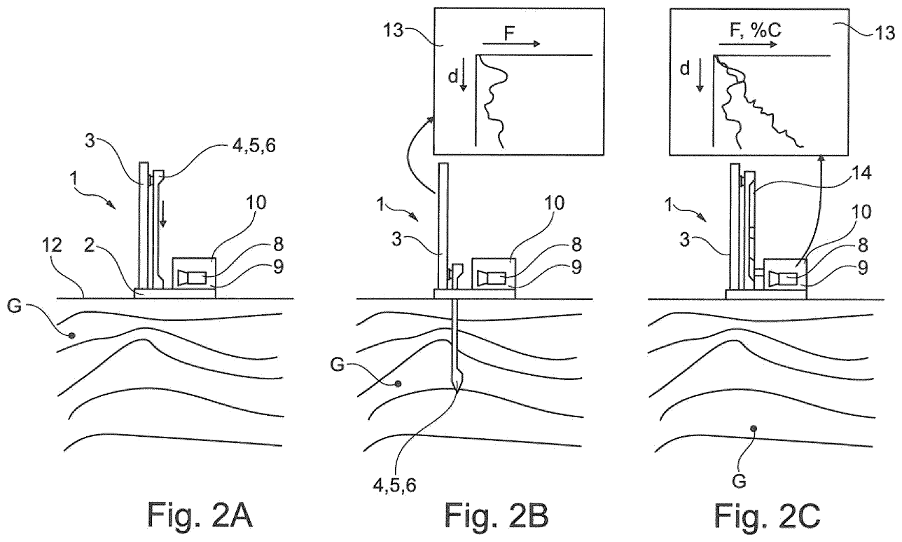

Under control of such a controller, the operation of the device 1 and an embodiment of the method according to the invention is further explained with reference to FIGS. 2A-2C.

In FIG. 2A, the device 1 is positioned with the base 2 onto a ground surface 12 of the ground G. The controller controls the guide column 3, such that the soil sampling tube 4, 5, 6 is inserted into the ground G. During this movement of the tube 4, 5, 6 into the ground G, the insertion force F is registered, such that a profile 13 relative to the depth d is obtained (see FIG. 2B)

After the predefined insertion depth is reached, the soil sampling tube 4, 5, 6 is retracted from the ground G by the guide column 3. During this retraction movement, the camera 8, LIBS 9 and Raman spectrometer 10 analyze the soil sample 14 at discrete positions via the cutaway portion 6 of the soil sampling tube 4, 5, 6.

In this example the carbon content of the soil sample 14 is analyzed and combined with the insertion force F into the profile 13.

Using the profile 13 of a number of soil samples performed over an area, it is possible, in combination with the accompanying geographical location determined by the GPS sensor 11, to determine the geological structure of said area.

With a method according to the invention, it would also be possible to determine the soil moisture profile over the length of the soil sample. If a soil sample would be transported as is done in the prior art, then the soil moisture profile would already be disturbed by dissipation of the moisture over the length of the soil sample and by evaporation of the moisture.

1. A method for obtaining geological information of a soil sample, which method comprises the steps of:
providing a soil sampling tube having a tip portion, a top portion positioned opposite the tip portion and along the length, between the tip portion and the top portion, a cutaway portion extending along a substantial part of the length of the tube;
inserting the soil sampling tube into the ground to a predefined depth, while registering the insertion force in combination with the insertion depth of the tip portion;
while retracting the soil sampling tube with a soil sample out of the ground, analyzing the elements in the soil sample at discrete positions starting adjacent to the top portion end and moving towards the tip portion end of the soil sample tube; and
combining the analyzing results per discrete position starting adjacent to the top portion end and moving towards the tip portion end with the registered insertion force for the insertion depth of the tip portion starting from-the insertion to the predefined depth.

2. The method according to claim 1, wherein the elements in the soil sample are analyzed using laser induced breakdown spectroscopy (LIBS) and/or Raman spectroscopy.

3. The method according to claim 2, wherein the elements are further analyzed by registering the gamma radiation for each discrete position.

4. The method according to claim 2, wherein the soil sample is further analyzed by a microwave sensor for determining the soil moisture.

5. The method according to claim 1, further comprising the step of removing an outer layer of the soil sample before the soil sample is analyzed.

6. The method according to claim 1, wherein the method further comprises the step of detecting a geographic position, combining the geographic position with the combined analyzing results and registered insertion force, and storing said combined information on a storage device.

7. A device for obtaining geological information of a soil sample according to the method of claim 1, which device comprises:
a base with a support surface for positioning the device with the support surface onto a ground surface;
a soil sampling tube having a tip portion, a top portion positioned opposite the tip portion and along the length, between the tip portion and the top portion, a cutaway portion extending along a substantial part of the length of the tube;
guide means arranged to the base and along which the soil sampling tube is guided in a direction perpendicular to the support surface; and
analyzing means arranged to the base, which analyzing means have a detecting area directed to the soil sampling tube.

8. The device according to claim 6, wherein the cutaway portion of the soil sampling tube faces the detecting area of the analyzing means.

9. The device according to claim 7, wherein the analyzing means comprises at least one of a laser induced breakdown spectroscope (LIBS) and a Raman spectroscope.

10. The device according to claim 8, wherein the analyzing means further comprise a gamma ray sensor and/or a microwave sensor.

11. The device according to claim 7, further comprising a camera, preferably a UV sensitive camera, arranged to the base and directed to the cutaway portion of the soil sampling tube.

12. The device according to claim 7, further comprising means for removing an outer layer of the sample, which removing means are arranged between the base and the analyzing means.
